(19) United States
(12) Patent Application Publication
(10) Pub. No.: US 2007/0014847 A1
(43) Pub. Date: Jan. 18, 2007

(54) Coated capsules and methods of making and using the same

(76) Inventors: Salah U. Ahmed, New City, NY (US); Sivakumar R. Vaithiyalingam, Parsippany, NJ (US); Sanjeev K. Gupta, Washington Township, NJ (US)

Correspondence Address: STERNE, KESSLER, GOLDSTEIN & FOX PLLC, 1100 NEW YORK AVENUE, N.W., WASHINGTON, DC, 20005, US

(21) Appl. No.: 11/480,574

(22) Filed: Jul. 5, 2006

Related U.S. Application Data
(60) Provisional application No. 60/695,856, filed on Jul. 5, 2005.

(51) Int. Cl. A61K 9/28; A61K 9/48; A61K 9/64

(52) U.S. Cl. 424/451; 424/456; 427/2.14

(57) ABSTRACT

The present invention relates to coated capsules with increased capsule shell pliability and resilience and methods of making the coated capsules. The present invention relates to methods of making coated capsules, the methods comprising applying a coating solution comprising sodium carboxymethylcellulose or sodium alginate to the exterior of a capsule to form a coating and drying the coating to produce a coated capsule.

This application claims the benefit of the filing date of U.S. Appl. No. 60/695,856, filed Jul. 5, 2005, which is incorporated by reference herein in its entirety.

### BACKGROUND OF THE INVENTION

1. Field of the Invention

The present invention relates to coated capsules with increased capsule shell pliability and resilience and methods of making the coated capsules. The present invention relates to methods of making coated capsules, the methods comprising applying a coating solution comprising sodium carboxymethylcellulose or sodium alginate to the exterior of a capsule to form a coating and drying the coating to produce a coated capsule.

2. Background Art

The encapsulation of medicinal agents remains a popular alternative to tablets or other methods of administering drugs. Capsules have numerous advantages, for example, they are tasteless, easily administered, and easily filled in large quantities commercially. Moreover, some people find it easier to swallow capsules than tablets and therefore prefer to take capsules whenever possible.

However, while capsules have advantages, encapsulation also has disadvantages. A pharmaceutical capsule generally consists of two parts, the cap and body, which are slipped over one another in part to seal the capsule. Additional procedures, such as banding or hermetic sealing, can be used to help prevent the capsule from separating at the seam between the cap and body thereby spilling its contents. This seam separation can be a problem when using large scale commercial capsule production methods because capsules tend to break during high speed counting processes or during packaging processes. Capsules can also break during storage, for example a single capsule can break in a bottle full of capsules, potentially coating nearby capsules with the capsule contents. Capsule breakage is especially problematic when the capsule contents are teratogenic, a controlled substance, or any other potentially hazardous material.

Others have tried to address the problem of capsule breakage or capsule sealing but have not come up with a cost effective method of sealing a pharmaceutical capsule to reduce capsule breakage thereby preventing the escape of the capsule's contents. Methods of sealing capsules using laser welding, methods of preventing tamper to capsules, methods of coating capsules, and coated capsules are described in U.S. Pat. Nos. 4,550,238, 4,643,894, 4,844,906, 4,973,480, 6,174,547, 6,183,466, 6,309,666, and 6,544,556. Similar methods and capsules are also described in US Publication Nos. 2003/0124182, 2003/0180362, 2004/0043064, 2004/0058000, 2004/0182283, and 2004/0204403. These patents and publications, however, do not solve the problem of capsule breakage by providing a capsule having increased capsule shell pliability and resilience.

Accordingly, there is a need in the art for the coated capsule of the present invention which has increased pliability and resilience to reduce capsule breakage thereby preventing the capsule contents from escaping.

### BRIEF SUMMARY OF THE INVENTION

The present invention is directed to a method of making a coated capsule, the method comprising: (a) applying a coating solution comprising sodium carboxymethylcellulose to the exterior of a capsule to form a coating and (b) drying the coating to produce a coated capsule, wherein the coated capsule is capable of being compressed up to about 20% of its original diameter without rupturing.

In some other embodiments, the present invention is directed to a method of making a coated capsule, the method comprising: (a) applying a coating solution comprising sodium alginate to the exterior of a capsule to form a coating; and (b) drying the coating to produce a coated capsule, wherein the coated capsule is capable of being compressed up to about 20% of its original diameter without rupturing.

In some embodiments, the capsule is selected from the group consisting of a gelatin capsule and a hydroxypropylmethylcellulose capsule. Optionally, the gelatin capsule can be a hard gelatin capsule.

The capsule of the present invention can be filled with, for example, a liquid, a powder, granules, microparticles, capsules, pellets, microtablets, tablets, or combinations thereof. In some embodiments, the capsule is filled with an active agent selected from the group consisting of a toxic drug, a teratogen, a controlled substance, a hormonal product, an androgen, isotretinoin, methadone, tretinoin, dutasteride, omeprazole, lansoprazole, and combinations thereof.

The coating solution of the present invention can further comprise an excipient selected from the group consisting of dextrose monohydrate, glycerin, lecithin, maltodextrin, a plasticizer, polyethylene glycol, sodium citrate, triethyl citrate, and combinations thereof. In some embodiments, the coating solution further comprises an aqueous solvent. Optionally, the coating solution is applied to the exterior of the capsule by spray coating.

In some embodiments, the drying is performed at a temperature of about 18° C. to about 35° C.

In some embodiments, the coating layer can be between about 5% to about 50% of the total capsule weight. In some embodiments, the coating layer is about 3% of the total capsule weight.

The present invention is also directed to a method of increasing capsule shell pliability, the method comprising: (a) applying a solution comprising sodium carboxymethylcellulose to the exterior of a capsule to form a coating and (b) drying the coating to produce a coated capsule with increased capsule shell pliability, wherein the coated capsule is capable of being compressed up to about 20% of its original diameter without rupturing. In some other embodiments, the present invention is also directed to a method of increasing capsule shell pliability, the method comprising: (a) applying a solution comprising sodium alginate to the exterior of a capsule to form a coating and (b) drying the coating to produce a coated capsule with increased capsule shell pliability, wherein the coated capsule is capable of being compressed up to about 20% of its original diameter without rupturing.

The present invention is also directed to a method of reducing capsule breakage, the method comprising: (a) applying a solution comprising sodium carboxymethylcellulose to the exterior of a capsule to form a coating and (b) drying the coating to produce a coated capsule with increased capsule shell pliability, wherein the coated capsule is capable of being compressed up to about 20% of its original diameter without rupturing. In some other embodiments, the present invention is also directed to a method of reducing capsule breakage, the method comprising: (a) applying a solution comprising sodium alginate to the exterior of a capsule to form a coating and (b) drying the coating to produce a coated capsule with increased capsule shell pliability, wherein the coated capsule is capable of being compressed up to about 20% of its original diameter without rupturing.

Further, the present invention is also directed to a method of reducing leakage of a liquid from a capsule, the method comprising: (a) applying a solution comprising sodium carboxymethylcellulose to the exterior of a capsule to form a coating and (b) drying the coating to produce a coated capsule with increased capsule shell pliability, wherein the coated capsule is capable of being compressed up to about 20% of its original diameter without rupturing. In some other embodiments, the method comprises: (a) applying a solution comprising sodium alginate to the exterior of a capsule to form a coating and (b) drying the coating to produce a coated capsule with increased capsule shell pliability, wherein the coated capsule is capable of being compressed up to about 20% of its original diameter without rupturing.

The present invention is also directed to coated capsules made using the methods of the present invention. In some embodiments, the coated capsules of the present invention are resilient. The present invention is also directed to coated capsules comprising a capsule and an exterior coating, wherein the exterior coating comprises sodium carboxymethylcellulose and maltodextrin.

In some embodiments, the exterior coating further comprises an excipient selected from the group consisting of dextrose monohydrate, glycerin, lecithin, maltodextrin, a plasticizer, polyethylene glycol, sodium citrate, triethyl citrate, and mixtures thereof. In some embodiments, the coated capsule comprises a capsule and an exterior coating, wherein the exterior coating comprises sodium carboxymethylcellulose and wherein the coated capsule is capable of being compressed up to about 20% of its original diameter without rupturing.

The present invention is also directed to kits comprising the coated capsules of the present invention. In some embodiments, the kit further comprises printed instructions for its use. In some embodiments, the kit further comprises a printed matter, a pre-recorded media device, or a planner describing the use of the coated capsules of the present invention to treat or prevent a condition or disease that can be treated using the coated capsules of the present invention.

The present invention is directed to a method of delivering the coated capsules of the present invention, to a patient in need thereof, the method comprising:
(a) registering in a computer readable storage medium identity of a physician permitted to prescribe the coated capsules;
(b) providing said patient with counseling information concerning a risk attendant to the coated capsules;
(c) obtaining informed consent of said patient to receive the coated capsules despite said risk;
(d) registering said patient in the computer readable medium after obtaining said informed consent; and
(e) permitting said patient access to the coated capsules.

In some embodiments of this method, the access to the coated capsule is a prescription.

Further, the present invention is also directed to a method of educating a consumer regarding the coated capsules of the present invention, the method comprising distributing the coated capsules to a consumer with consumer information at a point of sale.

### BRIEF DESCRIPTION OF THE DRAWINGS/FIGURES

FIG. 1 is a graph illustrating the increased pliability and resilience of a capsule of the present invention.

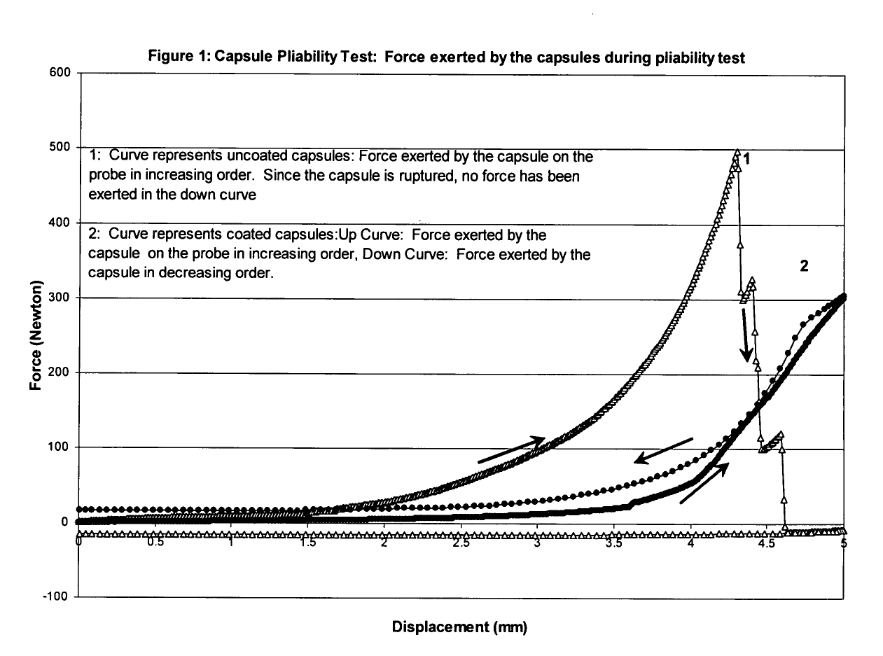

FIG. 2 provides an example of a process that can be used to prepare a capsule of the present invention.

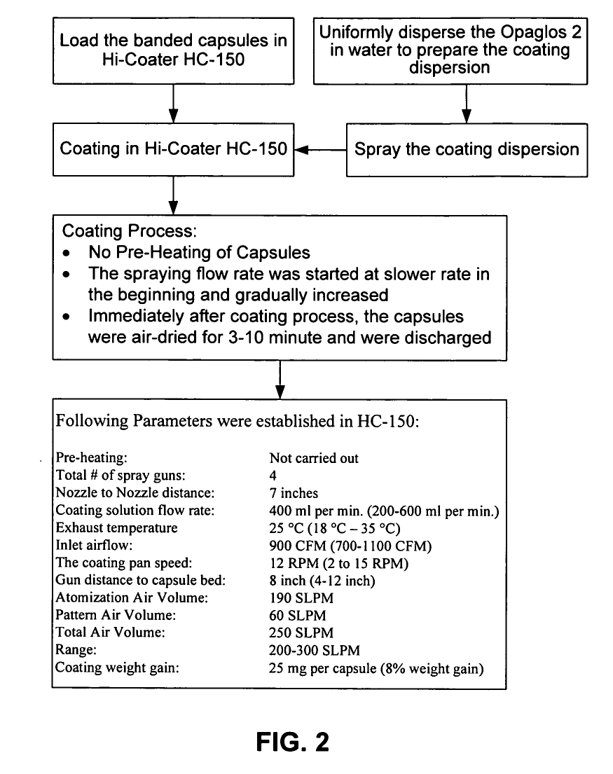

### DETAILED DESCRIPTION OF THE INVENTION

The present invention is directed to a method of making a coated capsule, the method comprising: (a) applying a coating solution comprising sodium carboxymethylcellulose to the exterior of a capsule to form a coating and (b) drying the coating to produce a coated capsule, wherein the coated capsule is capable of being compressed up to about 20% of its original diameter without rupturing.

In other embodiments, the present invention is directed to a method of making a coated capsule, the method comprising: (a) applying a coating solution comprising sodium alginate to the exterior of a capsule to form a coating and (b) drying the coating to produce a coated capsule, wherein the coated capsule is capable of being compressed up to about 20% of its original diameter without rupturing.

As one of skill in the art will appreciate, all descriptions to follow of the coated capsules, processes for making the coated capsules, and uses of the coated capsules will apply equally to those embodiments of the present invention which use sodium carboxymethylcellulose, sodium alginate, or mixtures thereof unless indicated otherwise.

“About” when used in conjunction with a percentage or other numerical amount means plus or minus 10% of that percentage or other numerical amount. For example, the term “about 80%” would encompass 80% plus or minus 8%.

Capsules come in many types, any of which is suitable for the present invention, but particularly suitable are the hard gelatin capsule, the soft elastic capsule, or the hydroxypropylmethylcellulose (HPMC) capsule. However, any capsule acceptable for pharmaceutical use is contemplated for use in the present invention.

The hard gelatin capsule consists of two sections. These two sections, the body and cap, slip over one another to form a sealed capsule. After the hard gelatin capsules have been filled and the cap has been applied, the capsule can be spot-welded or banded with molten gelatin at the seam to seal the capsule. Another approach used to solve capsule breakage is to use locking rings formed in both the cap and body to seal the capsule. This prevents the capsules from coming apart if subjected to vibration or rough handling, for example, as can occur during high speed counting processes or during packaging.

The soft elastic capsule is a soft, globular, gelatin shell that is thicker than the shell of hard gelatin capsules. They are also formed by overlapping two pieces, the cap and body, as described previously. The gelatin can be plasticized by adding, for example, glycerin, sorbitol or a similar polyol to form soft elastic capsules. This dosage form can be used with formulations where the suspending vehicle or solvent is an oil. However, the contents can be a liquid, powder, paste, or other form. These capsules are sealed at the seam to prevent them from breaking open prematurely.

The HPMC capsule is similar in structure and performs similarly to gelatin capsules in most applications. These capsules can be filled with powders, pellets, granules, liquids and semi-solids. HPMC capsules can be used in high-speed filling equipment and are generally suitable for any use indicated for gelatin capsules. HPMC capsules are low in moisture content making them ideal for hygroscopic formulations. HPMC capsules can be obtained from a variety of vendors but one example is the Quali-V® two piece HPMC capsule offered by Shionogi Qualicaps, (Whitsett, N.C.). As used herein, “capsule” means any pharmaceutically acceptable soluble container or shell which is capable of enclosing a substance, including, for example, gelatin or HPMC capsules.

A “coating solution” means a solution which is applied to the exterior of the capsule to form a coating.

A “coating” means a layer of a substance spread over a surface, e.g. a capsule. This coating can be applied for any reason known to one of skill in the art but, in particular, the present invention contemplates applying a coating to increase the pliability or elasticity of the capsule shell, to reduce capsule breakage, i.e. to increase capsule stability, to reduce leakage of liquid contents from the capsule, or to create a coated dosage form.

“Coated capsule” means a capsule as defined herein which has a coating as defined herein. For example, a coated capsule could be a hard gelatin capsule which has been coated with a coating solution and then dried.

A “pliable” capsule is a capsule capable of being bent, stretched, or compressed. For example, a “pliable” capsule can be compressed. In some embodiments, the pliable capsules of the present invention are also resilient.

A “resilient” capsule is a capsule capable of substantially resuming its original shape or position after being bent, stretched, or compressed. For example, a resilient capsule can be a capsule which substantially returns to its original shape after being compressed. In some embodiments, the capsules of the present invention are resilient. In some embodiments, the resilient capsules of the present invention substantially return to their original shape after compression without additional forces being exerted on them after the compressing force has been removed.

The “diameter” of a capsule (also the outside capsule diameter) refers to a straight line segment passing through the center of the capsule and terminating at the periphery of the capsule. In some embodiments, the diameter of the capsule is the thickness or width of the capsule.

In some embodiments, the coating solution used in the present invention comprises sodium carboxymethylcellulose. By way of non-limiting example, the coating solution can be prepared by placing an aqueous solvent in a stainless steel container with an air-operated mixer and a propeller type blade with vortex. The sodium carboxymethylcellulose can then be added slowly to the aqueous solvent to avoid flotation on the surface of the solvent. The mixing can continue until about 30 minutes after all the sodium carboxymethylcellulose has been added.

In other embodiments, the coating solution used in the present invention comprises an alginate component, such as sodium alginate. One of skill in the art will recognize that any suitable alginate component in the form of a water soluble salt of an alginic acid can also be used in the present invention. By way of non-limiting example, the coating solution can be prepared by placing an aqueous solvent in a stainless steel container with an air-operated mixer and a propeller type blade with vortex. The sodium alginate can then be added slowly to the aqueous solvent to avoid floatation on the surface of the solvent. The mixing can continue until about 30 minutes after all the sodium alginate has been added.

In some embodiments, the coating solution can further comprise an excipient such as, but not limited to, dextrose monohydrate, glycerin, lecithin, maltodextrin, a plasticizer, polyethylene glycol, sodium citrate, triethyl citrate, or mixtures thereof.

In some embodiments, the coating solution can comprise a mixture of sodium carboxymethylcellulose and sodium alginate.

The coating solution can be used to coat the capsules using any coating process known to one of skill in the art. In general, there are four major techniques for applying coatings to solid pharmaceutical dosage forms: 1) sugar coating, 2) film coating, 3) microencapsulation and 4) compression coating. In some embodiments, the present invention uses film coating techniques.

Film coating is the deposition of a thin polymeric film onto the dosage form from solutions that were initially organic-solvent based. However, the present invention uses film coating techniques which rely on inorganic solvents, such as water, as the prime solvent. Unlike some other coating techniques, film coating can be applied to substrates other than tablets, for example, powders, granules, or capsules.

The major components of a film coating formulation can include, but are not limited to, a polymer, a plasticizer, a colorant, and a solvent. Ideally the polymer is soluble in a wide range of solvent systems and is able to produce coatings with mechanical stability. Suitable polymers for film coating include, but are not limited to, cellulose ethers, particularly hydroxypropyl methylcellulose, hydroxypropyl cellulose, methylcellulose, sodium carboxymethylcellulose, and acrylics such as methacrylate or methyl methacrylate copolymers. In some embodiments, the polymer used in the coating solution is sodium carboxymethylcellulose. In some embodiments, the polymer used in the coating solution is sodium alginate. In some embodiments, the polymer used in the coating solution is a mixture of sodium carboxymethylcellulose and sodium alginate.

Optionally, a plasticizer can be added to the coating of the present invention to improve its flexibility. This can reduce the potential for film cracking and improve the adhesion of the film to the substrate, e.g. a capsule. Any plasticizer used in the formulation must show a high degree of compatibility with the selected polymer to achieve these benefits. Suitable plasticizers for the present invention include, but are not limited to, glycerin, propylene glycol, polyethylene glycols (e.g., PEG 400 or 900), triacetin, acetylated monoglyceride, citrate esters, and phthalate esters.

A coloring agent can optionally be added to the coating of the present invention to help identify the product. Any suitable coloring agent can be used including water-soluble dyes or insoluble pigments. However, in some embodiments, the present invention is a clear coating.

Suitable coating processes include coating pans and fluidized-bed coating equipment as described in Remington: The Science and Practice of Pharmacy, Lippincott Williams & Wilkins, 21st ed. (2005) (Remington's). In some embodiments, the coating solution is applied to the capsule using a spray coating technique. Either an air-less spray or an air spray coating technique can be used for film coating as described in Remington's. The use of a spray coating technique permits finely nebulized droplets of the coating solution to be delivered to the capsule surface. These techniques are ideal for commercial production because they ensure uniform coverage of the capsules without the adjacent capsules sticking together.

In some embodiments, a side-vented coating pan is used to apply the coating solution to the capsule by a spray coating technique. Suitable side-vented coating pans include the Accela-Cota (Thomas Engineering, Hoffman Estates, Ill.), the Fast Coater (O'Hara Manufacturing Ltd., Toronto Canada), the Hi-Coater (Vector Corp., Marion, Iowa), the Driacoater (Driam Metallprodukt, GmbH, Eriskirch, Germany), and the Pro Coater (Glatt Air Techniques, Ramsey, N.J.). In some embodiments, a Hi-Coater HCT-60 or HCT-150 can be used to coat the capsules with the exhaust temperature set to a temperature of about 18° C. to about 35° C. While not wishing to be bound to a specific theory, it is believed that performing the drying step at a temperature of about 18° C. to about 35° C. contributes to the unexpected increase in capsule shell pliability observed in the present invention.

In some embodiments, a commercial film coating material can be used to coat the capsule. For example, one suitable coating product is Opaglos 2® (Colorcon, West Point, Pa.).

In some embodiments, the capsule is filled with a liquid, a powder, granules, microparticles, capsules, pellets, microtablets, tablets, or combinations thereof. In some embodiments, the capsule is filled with an active agent selected from the group consisting of a toxic drug, a teratogen, a controlled substance, a hormonal product, an androgen, isotretinoin, methadone, tretinoin, dutasteride, omeprazole, lansoprazole, and mixtures thereof.

As used herein, a “toxic drug” means any substance used in the diagnosis, treatment, or prevention of a disease or as a component of a medication that is capable of causing injury or death, especially by chemical means.

As used herein, a “teratogen” means an agent, such as a virus, a drug, or radiation, that causes malformation of an embryo or fetus. In some embodiments, the teratogen is a drug. Some exemplary teratogens suitable for use in the present invention include: aminopterin, androgens, carbimazole, isotretinoin, methimazole, methotrexate, paramethadione, penicillamine, propylthiouracil, thalidomide, thiouracil, tretinoin, trimethadione, and mixtures thereof. However, one of ordinary skill in the art could substitute any teratogen as defined herein for use within the present invention.

As used herein, a “controlled substance” means a drug or chemical substance whose possession and use are regulated under the Controlled Substances Act (21 USC §§ 801-971). For example, a controlled substance means a drug or other substance, or immediate precursor, included in schedule I, II, III, IV, or V of part B of subchapter I of the Controlled Substances Act, herein incorporated by reference.

As used herein, a “hormonal product” is substance that contains a hormone. A “hormone,” as used herein, means a peptide or steroid produced by one tissue that can be conveyed via the bloodstream to another to effect physiological activity, such as growth or metabolism, or a synthetic compound that acts like a hormone in the body. In some embodiments, the capsule of the present invention can contain a hormonal product.

In some embodiments, the capsule can contain an antibiotic. For example, an antibiotic belonging to the beta-lactam family which includes penicillin. Penicillin is a highly prescribed antibiotic but it is also one of the most common drug allergies. Many individuals are either born with or develop an allergic reaction to penicillin or the other members of the beta-lactam family. These allergic reactions can range from hives, itchy eyes, and swelling of the lips, tongue or face to more severe reactions, such as anaphylaxis. The coated capsule of the present invention can be used to reduce penicillin containing capsule breakage thereby preventing individuals who may be allergic to penicillin from inadvertently coming into contact with the drug.

In some embodiments, the coated capsules of the present invention are capable of being compressed to about 20% of their original diameter without rupturing. For example, if a coated capsule of these embodiments of the present invention had an original diameter of 10 mm then this capsule would be capable of being compressed such that the capsule diameter when compressed is about 20% of the original 10 mm, i.e., about 2 mm. In some embodiments, following this compression the capsule will substantially return to its original shape including its original diameter.

In some embodiments, the coated capsules of the present invention are capable of being compressed to a diameter that is from about 20% to about 99% of their original diameter without rupturing. In some embodiments, the coated capsules of the present invention are capable of being compressed to a diameter that is from about 20% to about 95% of their original diameter without rupturing. In some embodiments, the coated capsules of the present invention are capable of being compressed to a diameter that is from about 20% to about 90% of their original diameter without rupturing. In some embodiments, the coated capsules of the present invention are capable of being compressed to a diameter that is from about 20% to about 85% of their original diameter without rupturing. In some embodiments, the coated capsules of the present invention are capable of being compressed to a diameter that is from about 20% to about 80% of their original diameter without rupturing. In some embodiments, the coated capsules of the present invention are capable of being compressed to a diameter that is from about 20% to about 75% of their original diameter without rupturing. In some embodiments, the coated capsules of the present invention are capable of being compressed to a diameter that is from about 20% to about 70% of their original diameter without rupturing. In some embodiments, the coated capsules of the present invention are capable of being compressed to a diameter that is from about 20% to about 65% of their original diameter without rupturing. In some embodiments, the coated capsules of the present invention are capable of being compressed to a diameter that is from about 20% to about 60% of their original diameter without rupturing. In some embodiments, the coated capsules of the present invention are capable of being compressed to a diameter that is from about 20% to about 55% of their original diameter without rupturing. In some embodiments, the coated capsules of the present invention are capable of being compressed to a diameter that is from about 20% to about 50% of their original diameter without rupturing.

The present invention is directed to a method of increasing capsule shell pliability, the method comprising: (a) applying a coating solution comprising sodium carboxymethylcellulose to the exterior of a capsule to form a coating and (b) drying the coating to produce a coated capsule with increased capsule shell pliability, wherein the coated capsule is capable of being compressed up to about 20% of its original diameter without rupturing.

In some embodiments, the present invention is directed to a method of increasing capsule shell pliability, the method comprising: (a) applying a coating solution comprising sodium alginate to the exterior of a capsule to form a coating and (b) drying the coating to produce a coated capsule with increased capsule shell pliability, wherein the coated capsule is capable of being compressed up to about 20% of its original diameter without rupturing.

This increase in capsule shell pliability is an unexpected benefit of coating a capsule with the coating solution of the present invention. As further described in the examples to follow, coating a capsule with a coating solution comprising sodium carboxymethylcellulose or sodium alginate can increase the pliability and elasticity of the capsule shell. This increased pliability will help reduce capsule breakage because, when a capsule is compressed, the capsules of the present invention will not rupture but will instead compress and then return to their original shape.

Accordingly, the present invention is directed to a method of reducing capsule breakage, the method comprising: (a) applying a coating solution comprising sodium carboxymethylcellulose to the exterior of a capsule to form a coating and (b) drying the coating to produce a coated capsule with increased capsule shell pliability, wherein the coated capsule is capable of being compressed up to about 20% of its original diameter without rupturing.

The present invention is also directed to a method of reducing capsule breakage, the method comprising: (a) applying a coating solution comprising sodium alginate to the exterior of a capsule to form a coating and (b) drying the coating to produce a coated capsule with increased capsule shell pliability, wherein the coated capsule is capable of being compressed up to about 20% of its original diameter without rupturing.

The present invention is also directed to a method of reducing leakage of a liquid from a capsule, the method comprising: (a) applying a solution comprising sodium carboxymethylcellulose to the exterior of a capsule to form a coating and (b) drying the coating to produce a coated capsule with increased capsule pliability, wherein the coated capsule is capable of being compressed up to about 20% of its original diameter without rupturing.

In other embodiments, the method of reducing leakage of a liquid from a capsule comprises: (a) applying a solution comprising sodium alginate to the exterior of a capsule to form a coating and (b) drying the coating to produce a coated capsule with increased capsule shell pliability, wherein the coated capsule is capable of being compressed up to about 20% of its original diameter without rupturing.

In some embodiments, the capsule coating covers 100% of the capsule surface area. In these embodiments, the coating functions not only to increase pliability of the capsule shell but also aids in reducing leakage of the contents from the capsule. The exterior coating will be liquid impermeable thereby preventing escape of any liquid contents in the capsule.

In some embodiments, the coating layer can be between about 5% to about 50% of the total capsule weight. In some embodiments, the coating layer can be between about 5% to about 25% of the total capsule weight. In some embodiments, the coating layer can be between about 5% to about 15% of the total capsule weight. In some embodiments, the coating layer is about 3% of the total capsule weight.

In some embodiments, the weight of the coating layer can be about 5% to about 100% of the weight of the empty capsule shell. In some embodiments, the weight of the coating layer can be about 10% to about 90% of the weight of the empty capsule shell. In some embodiments, the weight of the coating layer can be about 15% to about 85% of the weight of the empty capsule shell. In some embodiments, the weight of the coating layer can be about 20% to about 80% of the weight of the empty capsule shell. In some embodiments, the weight of the coating layer can be about 25% to about 75% of the weight of the empty capsule shell. In some embodiments, the weight of the coating layer can be about 30% to about 70% of the weight of the empty capsule shell. In some embodiments, the weight of the coating layer can be about 35% to about 65% of the weight of the empty capsule shell. In some embodiments, the weight of the coating layer can be about 40% to about 60% of the weight of the empty capsule shell.

The present invention is also directed to a pharmaceutical dosage form. The pharmaceutical dosage form of the present invention can be the product of any of the methods of the present invention. In some embodiments, the dosage form of the present invention comprises a pharmaceutically acceptable capsule which has been coated with a coating solution comprising sodium carboxymethylcellulose, sodium alginate, or mixtures thereof.

In some embodiments, the coating solution covering the exterior of the capsule can further comprise an excipient selected from the group consisting of dextrose monohydrate, glycerin, lecithin, maltodextrin, a plasticizer, polyethylene glycol, sodium citrate, triethyl citrate, and mixtures thereof.

The invention also provides kits comprising the coated capsules of the present invention. These kits can include one or more containers filled with one or more of the ingredients of the coated capsules of the invention.

In some embodiments, the kit comprises a container for the coated capsules of the present invention. Suitable containers include, for example, a bottle, a box, a blister card, a foil packet, or a combination thereof. Optionally, the kit also contains directions for properly administering the coated capsules. The kits can also be designed in a manner such that they are tamper resistant or designed to indicate if tampering has occurred. Optionally, the kit of the present invention can contain the coated capsules of the present invention in combination with another pharmaceutical composition.

Optionally associated with the container(s) in the kits of the present invention can be a notice or printed instructions. Such printed instructions can be in a form prescribed by a governmental agency regulating the manufacture, use or sale of pharmaceuticals or biological products, which notice reflects approval by the agency of the manufacture, use or sale for human administration to treat a condition or disease that can be treated using the coated capsules of the present invention. In some embodiments, the kit further comprises printed matter, which, e.g., provides information on the use of the coated capsules to treat a condition or disease or a pre-recorded media device which, e.g., provides information on the use of the coated capsule to treat a condition or disease, or a planner.

“Printed matter” can be, for example, one of a book, booklet, brochure or leaflet. The printed matter can describe the use of the coated capsules of the present invention to treat a condition or disease. Possible formats included, but are not limited to, a bullet point list, a list of frequently asked questions (FAQ) or a chart. Additionally, the information to be imparted can be illustrated in non-textual terms using pictures, graphics or other symbols.

“Pre-recorded media device” can be, for example, a visual media device, such as a videotape cassette, a DVD (digital video disk), filmstrip, 35 mm movie or any other visual media device. Alternately, pre-recorded media device can be an interactive software application, such as a CD-ROM (compact disk-read only memory) or floppy disk. Alternately, pre-recorded media device can be, for example, an audio media device, such as a record, audiocassette or audio compact disk. The information contained on the pre-recorded media device can describe the use of the coated capsules of the present invention to treat a condition or disease.

A “planner” can be, for example, a weekly, a monthly, a multi-monthly, a yearly, or a multi-yearly planner. The planner can be used as a diary to monitor dosage amounts, to keep track of dosages administered, or to prepare for future events wherein taking a regularly administered coated capsule of the present invention can be difficult. Alternately, the planner can be a calendar which will provide a means to monitor when a coated capsule has been taken and when it has not been taken. This type of planner will be particularly useful for patients having unusual schedules for administering medication to themselves. Additionally, the planner can be useful for the elderly, children, or other patient group who may administer medication to themselves and may become forgetful. One skilled in the art will appreciate the variety of planning tools that would be appropriate for use with the present invention.

The kit can also include a container for storing the other components of the kit. The container can be, for example, a bag, box, envelope or any other container that would be suitable for use in the present invention. In some embodiments, the container is large enough to accommodate each component and/or any administrative devices that may be accompany the coated capsules of the present invention. However, in some cases, it can be desirable to have a smaller container which can be hidden in a patient's pocketbook, briefcase or pocket.

In some embodiments, the present invention includes a kit comprising the coated capsules of the present invention. In some embodiments, the kit further comprises printed instructions for its use. In some embodiments, the kit further comprises a printed matter, a pre-recorded media device, or a planner describing the use of the coated capsules of the present invention to treat or prevent a condition or disease that can be treated using the coated capsules of the present invention.

In some aspects, the present invention provides a method of delivering the coated capsules of the present invention, to a patient in need thereof, the method comprising:
(a) registering in a computer readable storage medium identity of a physician permitted to prescribe the coated capsules;
(b) providing said patient with counseling information concerning a risk attendant to the coated capsules;
(c) obtaining informed consent of said patient to receive the coated capsules despite said risk;
(d) registering said patient in the computer readable medium after obtaining said informed consent; and
(e) permitting said patient access to the coated capsules.

In some embodiments of this method, the access to the coated capsules is a prescription.

The present invention is also directed to a method of educating a consumer regarding the coated capsules of the present invention, the method comprising distributing the coated capsules to a consumer with consumer information at a point of sale.

In some embodiments, the consumer information is presented in a format selected from the group consisting of: English language text, a foreign language text, a visual image, a chart, a telephone recording, a website, and access to a live customer service representative. In some embodiments, the consumer information is a direction for use, appropriate age use, indication, contraindication, appropriate dosing, warning, telephone number or website address.

In some embodiments, the method of educating the consumer further comprises providing professional information to a relevant person in a position to answer a consumer question regarding the coated capsules of the present invention. In some embodiments, the relevant person is a physician, physician assistant, nurse practitioner, pharmacist or customer service representative. In some embodiments, the distributing is to a location with a pharmacist or a health care provider.

All of the various embodiments or options described herein can be combined in any and all variations. Having generally described this invention, a further understanding can be obtained by reference to the examples provided herein. These examples are for purposes of illustration only and are not intended to be limiting.

### EXAMPLES

### Example 1

### Process for Preparing Capsule

The coated capsule of the present invention was prepared using the following exemplary method.

An active ingredient was wet milled with soybean oil to produce drug slurry. The formulation base was milled and mixed with drug slurry to obtain a final blend. Then a Liqfil F-40 automatic capsule filling machine was used for encapsulation, i.e., to fill the final blend in the body of the capsule and put the cap back on the body to lock. Next the capsules were weight sorted using a BOSCH 1500 weight sorter. A Hicapseal S-40 automatic capsule sealing machine was then used to band the capsules at the junction of the cap and body. The sealed capsules were visually inspected and stored in heat sealed aluminum bags until coating.

The coating dispersion was prepared by uniformly dispersing Opaglos 2 (sodium carboxymethylcellulose, maltodextrin, dextrose, lecithin, and sodium citrate) in water to form a uniform dispersion.

The sealed capsules were coated using a Hi-Coater HCT-150. The capsules were not preheated to preserve the moisture content of the capsules.

In the coating pan, 4 guns were used to atomize the coating solution at a flow rate of 200 to 600 ml per minute at a target rate of 400 ml per minute. The exhaust temperature was maintained at 25° C. (18° C.-35° C.). The airflow was maintained at 900 CFM (700-1100 CFM). The coating pan was kept at a speed of 12 RPM (2 to 15 RPM). Immediately after coating process, the capsules were air-dried for 3-10 minute and were discharged.

A weight gain of about 25 mg per capsule was achieved.

### Example 2

### Capsule Elasticity Testing

The elastic properties of the coated capsules of the present invention were tested and the results are contained in Table 1 below. A Texture Analyzer TA-XT Plus (Texture Technologies Corp., Scarsdale, N.Y.) was employed to analyze the brittleness and elasticity of both coated and uncoated (a control) hard gelatin capsules. The capsule was placed longitudinally on the fixed platform of the Texture Analyzer TA-XT Plus directly beneath the machine's probe. The probe was programmed to move at a rate of 2 mm per second and was lowered to press the probe against the capsule placed on the fixed platform. For these tests, the probe was a circular rod with a diameter of 0.25 inches.

The capsules tested (size 3) had an initial capsule diameter of 6.2 mm. The probe was set to travel 5 mm at a rate of 2 mm per second while the force required for such displacement due to resistance caused by the capsule was measured as a function of displacement distance. The uncoated capsule ruptured, exerting 498 Newton, when the probe pressed the capsule to 4.3 mm (69% of the initial capsule thickness). At this point, researchers observed visual rupturing of the capsule and content splattering. The force exerted on the probe from this point on remained zero.

In contrast, the coated capsule of the present invention did not rupture during the test. The probe traveled the full 5 mm test distance (80% of the initial capsule thickness) without rupturing the capsule. During the entire test, the capsule exerted force on the probe, including during the relaxation phase as the probe retracted from the 5 mm test distance. The capsule returned to its original shape following deformation during the test within about 2 to about 5 minutes after completing the test. The test results indicated that the coated capsule had increased pliability and resilience that could reduce the rate of accidental capsule rupture or capsule leakage.

TABLE 1
COMPRESSION AND RESILIENCE DATA

| | Force Required for | Force Required for |
|---|---|---|
| Distance Traveled by | Uncoated Capsule | Coated Capsule |
| Probe (Crushing) | (Newton) | (Newton) |
| 1.0 mm | 9.5 | 2.4 |
| 2.0 mm | 28.4 | 5.3 |
| 3.0 mm | 97 | 12.2 |
| 4.0 mm | 322 | 54 |
| 4.3 mm | 498 | 119 |
| 5.0 mm | 0 | 303 |
| | (Capsule ruptured due to | (Capsule did not |
| | brittleness) | rupture) |
| | Force exhibited by the | Force exhibited by the |
| Distance Traveled by | uncoated capsule on | coated capsule on the |
| Probe (Relaxation) | the probe (Newton) | probe (Newton) |
| 5.0 mm | 0 | 303 |
| 4.0 mm | 0 | 85.3 |
| 3.0 mm | 0 | 31 |
| 2.0 mm | 0 | 20 |
| 1.0 mm | 0 | 16.5 |

These examples illustrate possible methods of the present invention. While the invention has been particularly shown and described with reference to some embodiments thereof, it will be understood by those skilled in the art that they have been presented by way of example only, and not limitation, and various changes in form and details can be made therein without departing from the spirit and scope of the invention. Thus, the breadth and scope of the present invention should not be limited by any of the above-described exemplary embodiments, but should be defined only in accordance with the following claims and their equivalents.

All documents cited herein, including journal articles or abstracts, published or corresponding U.S. or foreign patent applications, issued or foreign patents, or any other documents, are each entirely incorporated by reference herein, including all data, tables, figures, and text presented in the cited documents.

What is claimed is:

1. A method of increasing capsule shell pliability, the method comprising:
(a) applying a coating solution comprising sodium carboxymethylcellulose to the exterior of a capsule to form a coating; and
(b) drying the coating to produce a coated capsule with increased capsule shell pliability;
wherein the coated capsule is capable of being compressed up to about 20% of its original diameter without rupturing.

2. The method of claim 1, wherein the capsule is selected from the group consisting of a gelatin capsule and a hydroxypropylmethylcellulose capsule.

3. The method of claim 2, wherein the gelatin capsule is a hard gelatin capsule.

4. The method of claim 1, wherein the capsule is filled with a liquid, a powder, granules, microparticles, capsules, pellets, microtablets, tablets, or combinations thereof.

5. The method of claim 1, wherein the capsule is filled with an active agent selected from the group consisting of a toxic drug, a teratogen, a controlled substance, a hormonal product, an androgen, isotretinoin, methadone, tretinoin, dutasteride, omeprazole, lansoprazole, and combinations thereof.

6. The method of claim 1, wherein the coating solution further comprises an excipient selected from the group consisting of dextrose monohydrate, glycerin, lecithin, maltodextrin, a plasticizer, polyethylene glycol, sodium citrate, triethyl citrate, and combinations thereof.

7. The method of claim 1, wherein the coating solution further comprises an aqueous solvent.

8. The method of claim 1, wherein the solution is applied to the exterior of the capsule by spray coating.

9. The method of claim 1, wherein the drying is performed at a temperature of about 18° C. to about 35° C.

10. A coated capsule made by the method of claim 1.

11. The coated capsule of claim 10, wherein the coated capsule is resilient.

12. A method of reducing capsule breakage, the method comprising:
(a) applying a coating solution comprising sodium carboxymethylcellulose to the exterior of a capsule to form a coating; and
(b) drying the coating to produce a coated capsule with increased capsule shell pliability;
wherein the coated capsule is capable of being compressed up to about 20% of its original diameter without rupturing.

13. The method of claim 12, wherein the coating solution further comprises an excipient selected from the group consisting of maltodextrin, dextrose monohydrate, lecithin and mixtures thereof.

14. A method of reducing leakage of a liquid from a capsule, the method comprising:
(a) applying a coating solution comprising sodium carboxymethylcellulose to the exterior of a capsule to form a coating; and
(b) drying the coating to produce a coated capsule with increased capsule shell pliability;
wherein the coated capsule is capable of being compressed up to about 20% of its original diameter without rupturing.

15. The method of claim 14, wherein the liquid comprises a component selected from a group consisting of a toxic drug, a teratogen, a controlled substance, a hormonal product or mixtures thereof.

16. The method of claim 15, wherein the teratogen is selected from the group consisting of aminopterin, androgens, carbimazole, isotretinoin, methimazole, methotrexate, paramethadione, penicillamine, propylthiouracil, thalidomide, thiouracil, tretinoin, trimethadione, and mixtures thereof.

17. The method of claim 14, wherein the coating solution further comprises an excipient selected from the group consisting of dextrose monohydrate, glycerin, lecithin, maltodextrin, a plasticizer, polyethylene glycol, sodium citrate, triethyl citrate, and mixtures thereof.

18. A coated capsule comprising a capsule and an exterior coating, wherein the exterior coating comprises sodium carboxymethylcellulose, wherein the coated capsule is capable of being compressed up to about 20% of its original diameter without rupturing.

19. The coated capsule of claim 18, wherein the exterior coating further comprises an excipient selected from the group consisting of dextrose monohydrate, glycerin, lecithin, maltodextrin, a plasticizer, polyethylene glycol, sodium citrate, triethyl citrate, and mixtures thereof.

20. A kit comprising the coated capsule of claim 18.

21. The kit of claim 20, further comprising printed instructions for its use.

22. The kit of claim 21, further comprising a printed matter describing the use of the coated capsule to treat a condition requiring delivery of the coated capsule, a pre-recorded media device describing the use of the coated capsule to treat a condition requiring delivery of the coated capsule, or a planner.

23. A method of increasing capsule shell pliability, the method comprising:
(a) applying a coating solution comprising sodium alginate to the exterior of a capsule to form a coating; and
(b) drying the coating to produce a coated capsule with increased capsule shell pliability;
wherein the coated capsule is capable of being compressed up to about 20% of its original diameter without rupturing.

24. The method of claim 23, wherein the capsule is selected from the group consisting of a gelatin capsule or a hydroxypropylmethylcellulose capsule.

25. The method of claim 23, wherein the capsule is filled with an active agent selected from the group consisting of a toxic drug, a teratogen, a controlled substance, a hormonal product, an androgen, isotretinoin, methadone, tretinoin, dutasteride, omeprazole, lansoprazole, and combinations thereof.

26. The method of claim 23, wherein the coating solution further comprises an excipient selected from the group consisting of dextrose monohydrate, glycerin, lecithin, maltodextrin, a plasticizer, polyethylene glycol, sodium citrate, triethyl citrate, and combinations thereof.

27. A coated capsule made by the method of claim 23.
